(19) United States
(12) Patent Application Publication
(10) Pub. No.: US 2005/0202083 A1
(43) Pub. Date: Sep. 15, 2005

(54) Storage stable tablets of fosinopril sodium

(76) Inventors: Pananchukunnath Manoj Kumar, Apartments (IN); Rajeev Shankar Mathur, Delhi (IN); Sanjeev Sethi, Haryana (IN); Rajiv Malik, Delhi (IN)

Correspondence Address: RANBAXY INC., 600 COLLEGE ROAD EAST, SUITE 2100, PRINCETON, NJ, 08540, US

(21) Appl. No.: 10/523,419

(22) PCT Filed: Aug. 1, 2003
(86) PCT No.: PCT/IB03/03113

§ 371 Date: Feb. 1, 2005

(51) Int. Cl. A61K 31/66; A61K 9/20

(52) U.S. Cl. 424/464; 514/102

(57) ABSTRACT

The technical field of the present invention relates to the selection of lubricants to provide a storage stable tablet of fosinopril, alone or in combination with a diuretic, as well as processes of preparation of the stable tablets. In particular, the lubricants are a combination of colloidal silicon dioxide and talc.

### FIELD OF THE INVENTION

The technical field of the present invention relates to the selection of lubricants to provide a storage stable tablet of fosinopril, alone or in combination with a diuretic, as well as processes of preparation of the stable tablets.

### BACKGROUND OF THE INVENTION

Fosinopril is the ester prodrug of the angiotensin converting enzyme inhibitor, fosinoprilat. Fosinopril alone or in combination with a diuretic, particularly a thiazide diuretic is indicated for the treatment of hypertension. It is also used as an adjunctive therapy for the management of heart failure as part of a conventional therapy that includes diuretics and, optionally, digitalis.

Fosinopril and its pharmaceutically acceptable salts, and in particular the sodium salt, have a low bulk density, poor flow characteristics, and a tendency to stick to metal surfaces. The combination of the above characteristics makes the manufacturing of tablets highly problematic and, in particular, makes necessary the careful selection and incorporation of suitable lubricants. In addition, the hydrolytic nature of fosinopril further complicates the selection of other pharmaceutically acceptable excipients.

In the past, conventional fosinopril sodium tablets were prepared using magnesium stearate as the lubricant. However, these tablets were highly sensitive to moisture and only marginally stable. Therefore, in order to achieve reasonable shelf lives these tablets required sophisticated protective packaging.

The above problem of selecting a suitable lubricant is addressed in U.S. Pat. No. 5,006,344 which discloses the use of either sodium stearyl fumarate or hydrogenated vegetable oil as the lubricant to prepare tablets with improved stability. Both of these compounds are long chain organic molecules. For example, according to the Handbook of Pharmaceutical Excipients. Third Edition (2000), the formula for sodium stearyl fumarate is C₂₂H₃₉NaO₄ and the formula for hydrogenated vegetable oil is R₁COOCH₃—CH(OOCR₂)—CH₂OOCR₃.

### SUMMARY OF THE INVENTION

In one general aspect there is provided a stable fosinopril tablet comprising fosinopril alone or in combination with a diuretic.

In another general aspect, there is provided a storage stable fosinopril tablet comprising fosinopril and a combination of colloidal silicon dioxide and talc.

Embodiments of the storage stable fosinopril tablet may include one or more of the following features. For example, the fosinopril may be one or more of free fosinopril acid and pharmaceutically acceptable salts of fosinopril. The pharmaceutically acceptable salt of fosinopril may be one or more of fosinopril sodium, fosinopril magnesium and fosinopril calcium. The pharmaceutically acceptable salt may be fosinopril sodium. The colloidal silicon dioxide may be from about 0.25% to about 10% by weight of the total tablet weight. The talc may be from about 0.25% to about 5% by weight of the total tablet weight.

The storage stable tablet may further include one or more pharmaceutically acceptable excipients and the one or more pharmaceutically acceptable excipients may be one or more of diluent, disintegrant, binder, coloring agent, and flavoring agent. The diluent may be one or more of calcium carbonate, calcium phosphate-dibasic, calcium phosphate-tribasic, calcium sulfate, cellulose-microcrystalline, cellulose powdered, dextrates, dextrins, dextrose excipients, fructose, kaolin, lactitol, lactose, mannitol, sorbitol, starch, starch pregelatinized, sucrose, sugar compressible and sugar confectioners, and in particular may be lactose. The binder may be one or more of methyl cellulose, hydroxypropyl cellulose, hydroxypropyl methylcellulose, polyvinylpyrrolidone, gelatin, gum arabic, ethyl cellulose, polyvinyl alcohol, pullulan, pregelatinized starch, agar, tragacanth, alginic acid derivatives and propylene glycol, and alginate, and in particular may be polyvinylpyrrolidone. The disintegrant may be one or more of low substituted hydroxypropyl cellulose, carboxymethyl cellulose, calcium carboxymethyl cellulose, sodium carboxymethyl cellulose, croscarmellose sodium, starch, crystalline cellulose, hydroxypropyl starch, and partly pregelatinized starch, and in particular may be croscarmellose sodium.

The storage stable tablet may further include one or more additional active ingredients and the one or more additional active ingredients may be a diuretic including one or more of chlorthalidone, flirosemide, triameterene, amiloride, spironolactone, and thiazide diuretics. The thiazide diuretic may be one or more of chlorothiazide, hydrochlorothiazide, flumethiazide and bendroflumethiazide, and the thiazide diuretic may be hydrochlorothiazide.

The one or more additional active ingredients also may be one or more of antidepressants, antidiabetics, antiulcers, analgesics, antihypertensives, antibiotics, antipsychotics, antineoplastics, antimuscarinics, diuretics, antimigraine agents, antivirals, anti-inflammatory agents, sedatives, antihistaminics, antiparasitic agents, antiepileptics and lipid lowering agents. The one or more additional active ingredients may be one or more of enalapril, captopril, benazepril, lisinopril, ranitidine, famotidine, ranitidine bismuth citrate, diltiazem, propranolol, verapamil, nifedipine, acyclovir, ciprofloxacin, simvastatin, atorvastatin, lovastatin, divalproex, venlafaxine, citalopram, paroxetine, selegiline, midazolam, fluoxetine, acarbose, buspirone, nimesulide, captopril, nabumetone, glimepiride, glipizide, etodolac, nefazodone and their pharmaceutically acceptable salts.

More than approximately 98% of an initial amount of fosinopril sodium may remain after storage for three months at 40° C. and 75% relative humidity as measured by high performance liquid chromatography. More than approximately 98% to 99% of an initial amount of fosinopril sodium may remain after storage for one week at 60° C. as measured by high performance liquid chromatography.

The storage stable table may include approximately 20% by weight of fosinopril sodium, approximately 45% by weight of anhydrous lactose, approximately 20% by weight of microcrystalline cellulose, approximately 3.5% by weight of crospovidone, approximately 5% by weight of polyvinylpyrrolidone, approximately 2.5% by weight of colloidal silicon dioxide, and approximately 4.0% by weight of talc.

In another general aspect, a storage stable fosinopril tablet includes from about 1% to about 40% by weight fosinopril sodium; up to 25% by weight of a diuretic; from about 20% to about 85% by weight of diluent; from about 1% to about 10% by weight of disintegrant; from about 1% to about 10% by weight of binder; from about 0.25% to about 10% by weight of colloidal silicon dioxide; and from about 0.25% to about 5% by weight of talc. The weights are percentages of the total tablet weight.

Embodiments of the storage stable fosinopril tablet may include any of the features described herein.

In another general aspect, a process for preparing storage stable fosinopril tablets includes the steps of (a) blending fosinopril in one or more of its free acid form and its pharmaceutically acceptable salts with one or more pharmaceutically acceptable excipients to form a blend, (b) optionally granulating the blend to form granules; (c) lubricating the blend or granules with colloidal silicon dioxide and talc; and (d) compressing into tablets.

Embodiments of the process may include any one of the features described herein. For example, the process may further include granulating the blend of step (a) and granulating the blend of step (a) may be a wet granulation process or a dry granulation process.

The blend of step (a) may further include one or more additional active ingredients. The additional active ingredient may be one or more of a diuretic comprising chlorthalidone, furosemide, triameterene, amiloride, spironolactone, and thiazide diuretics. The thiazide diuretic may be one or more of chlorothiazide, hydrochlorothiazide, flumethiazide and bendroflumethiazide, and in particular hydrochlorothiazide.

The process may further include using high performance liquid chromatography to measure the amount of fosinopril after storage. Greater than approximately 98% of an initial amount of fosinopril may remain after storage for three months at 40° C. and 75%, the amount of fosinopril being measured by high performance liquid chromatography. Greater than approximately 99% of an initial amount of fosinopril may remain after storage for one week at 60° C., the amount of fosinopril being measured by high performance liquid chromatography.

In another general aspect, a method for one or more of treating hypertension in a mammal and the management of heart failure as an adjunctive therapy in a mammal includes administering to the mammal one or more fosinopril tablets that include fosinopril in one or more of its free acid form and its pharmaceutically acceptable salts, colloidal silicon dioxide, and talc.

Embodiments of the method may include any one of the features described herein. For example, the tablet may further include a second active ingredient and the second active ingredient may be a diuretic including one or more of chlorthalidone, furosemide, triameterene, amiloride, spironolactone, and thiazide diuretics. The thiazide diuretic comprises one or more of chlorothiazide, hydrochlorothiazide, flumethiazide and bendroflumethiazide and in particular the thiazide diuretic may be hydrochlorothiazide.

Greater than approximately 98% of an initial amount of fosinopril may remain after storage for three months at 40° C. and 75% relative humidity as measured by high performance liquid chromatography. Greater than approximately 99% of an initial amount of fosinopril sodium remains after storage for one week at 60° C. as measured by high performance liquid chromatography.

In another general aspect there is provided a stable fosinopril tablet comprising fosinopril, and a combination of colloidal silicon dioxide and talc as lubricant wherein colloidal silicon dioxide may vary from about 0.25% to about 10% by weight and talc about 0.25% to about 5% by weight, of the total tablet weight.

In another general aspect there is provided a stable fosinopril tablet comprising fosinopril, a diuretic, and a combination of colloidal silicon dioxide and talc as lubricant wherein colloidal silicon dioxide may vary from about 0.25% to about 10% by weight and talc about 0.25% to about 5% by weight, of the total tablet weight.

In another general aspect there is provided a method for the management of heart failure in a mammal by administering as adjunctive therapy to the said mammal fosinopril tablet comprising fosinopril, a diuretic, colloidal silicon dioxide and talc.

The details of one or more embodiments of the inventions are set forth in the description below. Other features, objects, and advantages of the invention will be apparent from the description and claims.

### DETAILED DESCRIPTION OF THE INVENTION

The inventors have now discovered that the use of a combination of talc and colloidal silicon dioxide in the formulation functions surprisingly well as lubricants during the tableting process and further surprisingly increases the stability of the tablet and provides reasonably long shelf lives for the thus formed fosinopril tablets. Advantageously, these benefits result even at low concentrations of the colloidal silicon dioxide and talc. In contrast to the very long chain organic molecules described above as lubricants, the Handbook of Pharmaceutical Excipients, Third Edition (2000) describes colloidal silicon dioxide and talc as inorganic molecules that have, for example, the molecular formulae SiO₂ and Mg₆(Si₂O₅)₄(OH)₄, respectively, although for purposes of the inventions described herein, variations and other forms of silicon dioxide and talc are intended to be encompassed within these terms.

Conventionally, colloidal silicon dioxide and talc are used as glidants. However, to the inventors' surprise the combination of the two showed excellent lubricant properties and, relevant to problems in formulating dosage forms of fosinopril, the tablets thus prepared had improved shelf stability. Colloidal silicon dioxide or talc used individually in higher concentrations may also provide proper lubrication during processing of tablets and stability during storage. However, moving to higher concentrations of lubricant increases the tablet weight and may also exceed the permissible daily intake of the lubricant. Further, the higher concentration of lubricant may also hamper the dissolution of the drug from the tablets and consequently the bioavailability of the drug. The combination of colloidal silicon dioxide and talc, on the other hand, appear to have a synergistic action and is therefore effective in reasonably low amounts. When used in combination, the amount of talc may vary from about 0.25% to about 5% by weight and the amount of colloidal silicon dioxide may vary from about 0.25% to about 10% by weight with respect to the total weight of tablet.

Comparative stability results were generated under two conditions: (1) at 40° C. and 75% relative humidity over a period of three months (see Table 1, below) and (2) at 60° C. for one week (see Table 2, below). The results were generated for fosinopril tablets that included a combination of colloidal silicon dioxide and talc as lubricants and for the marketed Monopril® tablets. The results indicate the benefits of using a combination of colloidal silicon dioxide and talc as the fosinopril tablet lubricant.

The term “fosinopril” as used herein includes both the free acid form as well as its pharmaceutically acceptable salts, such as those with sodium, magnesium, calcium, etc. In particular, fosinopril sodium may be used. The concentration of fosinopril sodium may vary from about 1% to about 40% by weight of the total tablet weight.

Stable fosinopril tablets may also include a second active ingredient, and particularly may include a diuretic. Examples of suitable diuretics include chlorthalidone, furosemide, triameterene, amiloride, spironolactone, thiazide diuretics, and the like. Suitable examples of thiazide diuretics include chlorthiazide, hydrochlorthiazide, flumethiazide, bendroflumethiazide, and the like. The concentration of diuretic may vary up to about 25% by weight of the total tablet weight.

In addition to the tablet including fosinopril as an active ingredient, and colloidal silicon dioxide and talc as the lubricant, fosinopril tablets may also include pharmaceutically acceptable excipients. The term “pharmaceutically acceptable inert excipients” as used herein includes all excipients used in the art of manufacturing tablets.

Examples of pharmaceutically acceptable excipients include binders, diluents, disintegrants, coloring agents, flavoring agents, and the like.

Examples of suitable diluents include calcium carbonate, calcium phosphate-dibasic, calcium phosphate-tribasic, calcium sulfate, cellulose-microcrystalline, cellulose powdered, dextrates, dextrins, dextrose excipients, fructose, kaolin, lactitol, lactose, mannitol, sorbitol, starch, starch pregelatinized, sucrose, sugar compressible, sugar confectioners, and the like. The concentration of diluent may vary from about 20% to about 85% by weight of the total tablet weight.

Examples of suitable binders include methyl cellulose, hydroxypropyl cellulose, hydroxypropyl methylcellulose, polyvinylpyrrolidone, gelatin, gum arabic, ethyl cellulose, polyvinyl alcohol, pullulan, pregelatinized starch, agar, tragacanth, alginic acid derivatives, propylene glycol and the like. The concentration of binder may vary from about 1% to about 10% by weight of the total tablet weight.

Examples of suitable disintegrants include low substituted hydroxypropyl cellulose, carboxymethyl cellulose, calcium carboxymethyl cellulose, sodium carboxymethyl cellulose, croscarmellose sodium A-type (Ac-di-sol), starch, crystalline cellulose, hydroxypropyl starch, partly pregelatinized starch, and the like. The concentration of disintegrant may vary from about 1% to about 10% by weight of the total tablet weight.

Suitable coloring agents and flavoring agents include FDA approved colors and flavors for oral use that are compatible with the other ingredients of the tablet.

The stable fosinopril tablet may be prepared by processes known in the pharmaceutical arts, for example, by comminuting, mixing, granulation, melting, sizing, filling, drying, molding, immersing, coating, compressing, etc. Examples of specific processes suitable for preparing the tablets include wet granulation, dry granulation and direct compression.

In one of the processing embodiments, a wet granulation process of preparing the tablets includes the steps of blending fosinopril sodium and, optionally, a diuretic with one or more diluents and disintegrants; granulating the blend with a solution/dispersion of one or more binders in one or more suitable solvents; drying and sieving the granules; lubricating the blend with talc and colloidal silicon dioxide; and compressing into tablets. Specific examples of suitable solvents for preparing the solution/dispersion of binder include one or more of methylene chloride, isopropyl alcohol, acetone, methanol, ethanol, water and the like.

In another processing embodiment, a direct compression process for preparing the tablets includes the steps of blending fosinopril sodium and, optionally, a diuretic with one or more diluents, disintegrants and binders; lubricating the blend with talc and colloidal silicon dioxide; and compressing into tablets.

In another processing embodiment, a dry granulation process for preparing the tablets includes the steps of blending fosinopril sodium and, optionally, a diuretic with one or more diluents, binders and disintegrants; dry granulating the blend by slugging or roller compaction; lubricating the blend with talc and colloidal silicon dioxide; and compressing into tablets.

The fosinopril tablets prepared by any of the above methods may optionally be coated with one or more functional and/or non-functional coatings, if desired.

The following examples further exemplify the inventions and are not intended to limit the scope of the inventions.

### FORMULATIONS OF EXAMPLES 1-3

| | Amount (mg/tablet) | | |
|---|---|---|---|
| Ingredients | Example 1 | Example 2 | Example 3 |
| Fosinopril Sodium | 40.0 | 20.0 | 20.0 |
| Hydrochlorthaizide | — | 12.5 | 12.5 |
| Anhydrous Lactose | 90.0 | 32.5 | 97.5 |
| Microcrystalline Cellulose | 40.0 | 20.0 | 40.0 |
| Crospovidone | 7.0 | 3.5 | 7.0 |
| Polyvinylpyrrolidone | 10.0 | 5.0 | 10.0 |
| Colloidal Silicon Dioxide | 5.0 | 2.5 | 5.0 |
| Talc | 8.0 | 4.0 | 8.0 |
| Isopropyl Alcohol | qs | qs | qs |

Process:
1. Fosinopril sodium and hydrochlorthiazide (Examples 2 and 3) were blended with lactose, microcrystalline cellulose and a portion of the crospovidone.
2. The blend of step 1 was granulated with polyvinylpyrrolidone solution in isopropyl alcohol.
3. The granules obtained above were dried and blended with crospovidone, talc and colloidal silicon dioxide.
4. The lubricated blend of step 3 was compressed into suitably sized tablets.

As evident from the formulation data above, a tablet according to Example 1 includes approximately 20% by weight of fosinopril sodium, approximately 45% by weight of anhydrous lactose, approximately 20% by weight of microcrystalline cellulose, approximately 3.5% by weight of crospovidone, approximately 5% by weight of polyvinylpyrrolidone, approximately 2.5% by weight of colloidal silicon dioxide, and approximately 4.0% by weight of talc. Fosinopril tablets prepared with the formulation of Example 1 were analyzed for the initial amount of fosinopril sodium using an in-house validated high performance liquid chromatography (HPLC) method. One batch of these samples was then kept at 40° C. and 75% relative humidity for three months. The amount of fosinopril sodium at the end of the first, the second and the third month was analyzed to determine the amount of fosinopril sodium; the results obtained are listed in Table 1. Another batch of these samples was kept at 60° C. for one week and then was analyzed to determine the amount of fosinopril sodium; the results obtained at one week are listed in Table 2.

TABLE 1
Stability results generated after storage at 40° C. and 75%
relative humidity

| | Amount of Fosinopril sodium (mg) | | | |
|---|---|---|---|---|
| | | After 1 month | After 2 month | After 3 month |
| | | at 40° C. and | at 40° C. and | at 40° C. and |
| | | 75% relative | 75% relative | 75% relative |
| Tablets | Initial | humidity | humidity | humidity |
| Fosinopril | 40 | 39.69 | 39.98 | 39.23 |
| tablets | | | | |
| prepared per | | | | |
| Example 1 | | | | |
| “MONOPRIL” | 40.01 | — | — | 39.95 |
| Commercially | | | | |
| available | | | | |
| fosinopril | | | | |
| sodium | | | | |
| tablets | | | | |
| (strength - | | | | |
| 40 mg) of | | | | |
| BRISTOL | | | | |
| MYERS | | | | |
| SQUIBB | | | | |

TABLE 2
Stability results generated after storage at 60° C.

| | Amount of Fosinopril | |
|---|---|---|
| | sodium (mg) | |
| Tablets | Initial | After one week at 60° C. |
| Fosinopril tablets prepared as per the | 40.01 | 39.95 |
| Example 1 | | |
| “MONOPRIL” Commercially available | 40.01 | 39.79 |
| fosinopril sodium tablets (strength - | | |
| 40 mg) of BRISTOL MYERS SQUIBB | | |

As evident from the above stability data, after three months storage at 40C and 75% relative humidity, the fosinopril sodium tablets made according to the formulation of Example 1 were stable, e.g., greater than approximately 98% of the fosinopril sodium remained. Similarly, after one week storage at 60C, the fosinopril sodium tablets made according to the formulation of Example 1 were stable, e.g., greater than approximately 99% of the fosinopril sodium remained.

While several particular forms of the inventions have been described, it will be apparent that various modifications and combinations of the inventions detailed in the text can be made without departing from the spirit and scope of the inventions. For example, the combination of colloidal silicon dioxide and talc can be used as a lubricant with any active pharmaceutical ingredient that is chemically and physically compatible with colloidal silicon dioxide and talc. Examples of classes of active pharmaceutical ingredients that may be used with the combination lubricant described here include antidepressants, antidiabetics, antiulcers, analgesics, antihypertensives, antibiotics, antipsychotics, antineoplastics, antimuscarinics, diuretics, antimigraine agents, antivirals, anti-inflammatory agents, sedatives, antihistaminics, antiparasitic agents, antiepileptics and lipid lowering agents. Illustrative examples of specific drugs of the above classes include enalapril, captopril, benazepril, lisinopril, ranitidine, famotidine, ranitidine bismuth citrate, diltiazem, propranolol, verapamil, nifedipine, acyclovir, ciprofloxacin, simvastatin, atorvastatin, lovastatin, divalproex, venlafaxine, citalopram, paroxetine, selegiline, midazolam, fluoxetine, acarbose, buspirone, nimesulide, captopril, nabumetone, glimepiride, glipizide, etodolac, nefazodone and their pharmaceutically acceptable salts. Further, it is contemplated that any single feature or any combination of optional features of the inventive variations described herein may be specifically excluded from the claimed inventions and be so described as a negative limitation. Accordingly, it is not intended that the inventions be limited, except as by the appended claims.

1. A storage stable fosinopril tablet comprising fosinopril and a combination of colloidal silicon dioxide and talc.

2. The storage stable tablet of claim 1, wherein the fosinopril comprises one or more of free fosinopril acid and pharmaceutically acceptable salts of fosinopril.

3. The storage stable tablet of claim 2, wherein the pharmaceutically acceptable salt of fosinopril comprises one or more of fosinopril sodium, fosinopril magnesium and fosinopril calcium.

4. The storage stable tablet of claim 3, wherein the pharmaceutically acceptable salt comprises fosinopril sodium.

5. The storage stable tablet of claim 1, wherein the colloidal silicon dioxide comprises from about 0.25% to about 10% by weight of the total tablet weight.

6. The storage stable tablet of claim 1, wherein the talc comprises from about 0.25% to about 5% by weight of the total tablet weight.

7. The storage stable tablet of claim 1, wherein the tablet further comprises one or more pharmaceutically acceptable excipients.

8. The storage stable tablet of claim 7, wherein the one or more pharmaceutically acceptable excipients comprise one or more of diluent, disintegrant, binder, coloring agent, and flavoring agent.

9. The storage stable tablet of claim 8, wherein the diluent comprises one or more of calcium carbonate, calcium phosphate-dibasic, calcium phosphate-tribasic, calcium sulfate, cellulose-microcrystalline, cellulose powdered, dextrates, dextrins, dextrose excipients, fructose, kaolin, lactitol, lactose, mannitol, sorbitol, starch, starch pregelatinized, sucrose, sugar compressible and sugar confectioners.

10. The storage stable tablet of claim 9, wherein the diluent comprises lactose.

11. The storage stable tablet of claim 8, wherein the binder comprises one or more of methyl cellulose, hydroxypropyl cellulose, hydroxypropyl methylcellulose, polyvinylpyrrolidone, gelatin, gum arabic, ethyl cellulose, polyvinyl alcohol, pullulan, pregelatinized starch, agar, tragacanth, alginic acid derivatives and propylene glycol, and alginate.

12. The storage stable tablet of claim 11, wherein the binder comprises polyvinylpyrrolidone.

13. The storage stable tablet of claim 8, wherein the disintegrant comprises one or more of low substituted hydroxypropyl cellulose, carboxymethyl cellulose, calcium carboxymethyl cellulose, sodium carboxymethyl cellulose, croscarmellose sodium, starch, crystalline cellulose, hydroxypropyl starch, and partly pregelatinized starch.

14. The storage stable tablet of claim 13, wherein the disintegrant comprises croscarmellose sodium.

15. The storage stable tablet of claim 1, wherein the tablet further comprises one or more additional active ingredients.

16. The storage stable tablet of claim 15, wherein the one or more additional active ingredients comprise a diuretic comprising one or more of chlorthalidone, furosemide, triameterene, amiloride, spironolactone, and thiazide diuretics.

17. The storage stable tablet of claim 16, wherein the thiazide diuretic comprises one or more of chlorothiazide, hydrochlorothiazide, flumethiazide and bendroflumethiazide.

18. The storage stable tablet of claim 17 wherein the thiazide diuretic comprises hydrochlorothiazide.

19. The storage stable tablet of claim 15, wherein the one or more additional active ingredients comprise one or more of antidepressants, antidiabetics, antiulcers, analgesics, antihypertensives, antibiotics, antipsychotics, antineoplastics, antimuscarinics, diuretics, antimigraine agents, antivirals, anti-inflammatory agents, sedatives, antihistaminics, antiparasitic agents, antiepileptics and lipid lowering agents.

20. The storage stable tablet of claim 15, wherein the one or more additional active ingredients comprise one or more of enalapril, captopril, benazepril, lisinopril, ranitidine, famotidine, ranitidine bismuth citrate, diltiazem, propranolol, verapamil, nifedipine, acyclovir, ciprofloxacin, simvastatin, atorvastatin, lovastatin, divalproex, venlafaxine, citalopram, paroxetine, selegiline, midazolam, fluoxetine, acarbose, buspirone, ninesulide, captopril, nabumetone, glimepiride, glipizide, etodolac, nefazodone and their pharmaceutically acceptable salts.

21. The storage stable tablet of claim 1, wherein greater than approximately 98% of an initial amount of fosinopril sodium remains after storage for three months at 40° C. and 75% relative humidity as measured by high performance liquid chromatography.

22. The storage stable tablet of claim 1, wherein greater than approximately 98% of an initial amount of fosinopril sodium remains after storage for one week at 60° C. as measured by high performance liquid chromatography.

23. The storage stable tablet of claim 22, wherein greater than approximately 99% of an initial amount of fosinopril sodium remains after storage for one week at 60° C. as measured by high performance liquid chromatography.

24. The storage stable tablet of claim 1, wherein greater than approximately 98% of an initial amount of fosinopril sodium remains after storage for one week at 60° C. as measured by high performance liquid chromatography.

25. The storage stable table of claim 7, wherein the tablet comprises approximately 20% by weight of fosinopril sodium, approximately 45% by weight of anhydrous lactose, approximately 20% by weight of microcrystalline cellulose, approximately 3.5% by weight of crospovidone, approximately 5% by weight of polyvinylpyrrolidone, approximately 2.5% by weight of colloidal silicon dioxide, and approximately 4.0% by weight of talc.

26. A storage stable fosinopril tablet comprising:
from about 1% to about 40% by weight fosinopril sodium;
up to 25% by weight of a diuretic;
from about 20% to about 85% by weight of diluent;
from about 1% to about 10% by weight of disintegrant;
from about 1% to about 10% by weight of binder;
from about 0.25% to about 10% by weight of colloidal silicon dioxide; and
from about 0.25% to about 5% by weight of talc,
wherein the weights are percentages of the total tablet weight.

27. A process for preparing storage stable fosinopril tablets, the process comprising the steps of:
a. blending fosinopril in one or more of its free acid form and its pharmaceutically acceptable salts with one or more pharmaceutically acceptable excipients to form a blend,
b. optionally granulating the blend to form granules;
c. lubricating the blend or granules with colloidal silicon dioxide and talc; and
d. compressing into tablets.

28. The process according to claim 27, further comprising granulating the blend of step (a).

29. The process according to claim 28, wherein granulating the blend of step (a) comprises a wet granulation process.

30. The process according to claim 28, wherein granulating the blend of step (a) comprises a dry granulation process.

31. The process according to claim 27, wherein the blend of step (a) further comprises one or more additional active ingredients.

32. The process according to claim 31, wherein the additional active ingredient comprises one or more of a diuretic comprising chlorthalidone, furosemide, triameterene, amiloride, spironolactone, and thiazide diuretics.

33. The process according to claim 32, wherein the thiazide diuretic comprises one or more of chlorothiazide, hydrochlorothiazide, flumethiazide and bendroflumethiazide.

34. The process according to claim 33, wherein the thiazide diuretic comprises hydrochlorothiazide.

35. The process according to claim 27, further comprising using high performance liquid chromatography to measure the amount of fosinopril after storage.

36. The process according to claim 35, wherein greater than approximately 98% of an initial amount of fosinopril remains after storage for three months at 40° C. and 75%, the amount of fosinopril being measured by high performance liquid chromatography.

37. The process according to claim 35, wherein greater than approximately 99% of an initial amount of fosinopril remains after storage for one week at 60° C., the amount of fosinopril being measured by high performance liquid chromatography.

38. A method for one or more of treating hypertension in a mammal and the management of heart failure as an adjunctive therapy in a mammal, the method comprising administering to the mammal one or more fosinopril tablets comprising fosinopril in one or more of its free acid form and its pharmaceutically acceptable salts, colloidal silicon dioxide, and talc.

39. The method according to claim 38, wherein the tablet further comprises a second active ingredient.

40. The method according to claim 39, wherein the second active ingredient comprises a diuretic comprising one or more of chlorthalidone, furosemide, triameterene, amiloride, spironolactone, and thiazide diuretics.

41. The method according to claim 40, wherein the thiazide diuretic comprises one or more of chlorothiazide, hydrochlorothiazide, flumethiazide and bendroflumethiazide.

42. The method according to claim 41, wherein the thiazide diuretic comprises hydrochlorothiazide.

43. The method according to claim 38, wherein greater than approximately 98% of an initial amount of fosinopril remains after storage for three months at 40° C. and 75% relative humidity as measured by high performance liquid chromatography.

44. The storage stable tablet of claim 38, wherein greater than approximately 99% of an initial amount of fosinopril sodium remains after storage for one week at 60° C. as measured by high performance liquid chromatography.
